(19) United States
(12) Patent Application Publication
(10) Pub. No.: US 2026/0250180 A1
(43) Pub. Date: Aug. 27, 2026

(54) METHOD FOR CHARACTERIZING THE EFFECTIVE DIELECTRIC CONSTANT OF SUBSTRATE INCLUDING GLASS FIBERS

(71) Applicant: JCET Management Co., LTD., Shanghai (CN)

(72) Inventors: Wei LIN, Shanghai (CN); Fan HU, Shanghai (CN); Yanbo TANG, Shanghai (CN); Yanjia SHI, Shanghai (CN); Cheng YANG, Shanghai (CN); Boping WU, Shanghai (CN)

(73) Assignee: JCET Management Co., LTD., Shanghai (CN)

(21) Appl. No.: 19/537,167

(22) Filed: Feb. 11, 2026

(30) Foreign Application Priority Data
Feb. 26, 2025 (CN) 202510215400.2

(51) Int. Cl. C03C 3/087; H01P 3/08

(52) U.S. Cl. CPC C03C 3/087; H01P 3/08

(57) ABSTRACT

A method for characterizing an effective dielectric constant of a substrate including glass fibers, including: constructing a real model, where the real model includes: the substrate, a glass fiber-woven layer on the substrate, and a transmission line trace on the substrate; performing a phase shift simulation on the real model according to phase shift value; and obtaining first information; constructing a simplified model, where the simplified model includes: the substrate, and the transmission line trace on the substrate; setting a dielectric constant of the substrate as a variable; performing parameter scanning simulation; and obtaining a second information; and according to the first and second information, determining a dielectric constant value when the phase shift value of the simplified model equals the phase shift value of the real model, and using the dielectric constant value as the effective dielectric constant of the substrate including the glass fibers.

### CROSS-REFERENCE TO RELATED APPLICATIONS

This application claims the benefit of priority to Chinese Application No. 202510215400.2, filed on February 26, 2025, which is incorporated herein by reference in its entirety.

### TECHNICAL FIELD

The present disclosure relates to the field of integrated circuits, and particularly relates to a method for characterizing the effective dielectric constant of a substrate including glass fibers, an apparatus, and a computer-readable storage medium.

### BACKGROUND

Substrates including glass fibers play a critical role in the development and application of modern electronic products, particularly in the chip package industry. The substrates including glass fibers are glass fiber-woven structures formed by glass fiber cloth embedded in a resin base material, and the resin base material may be epoxy or BT resin. This makes substrates including glass fibers have the following advantages: high mechanical strength, excellent thermal stability, and superior electrical performance.

### SUMMARY

The present disclosure provides a method for characterizing the effective dielectric constant of a substrate including glass fibers, an apparatus, and a computer-readable storage medium.

The method for characterizing the effective dielectric constant of a substrate including glass fibers, including: simulation of an actual truncated trace: constructing a real model, the real model includes a substrate, a glass fiber-woven layer arranged within the substrate, and a transmission line trace arranged on the substrate; performing phase shift value simulation on the real model and obtaining the relationship curve of frequency with the phase shift value; simulation of a simplified model: constructing a simplified model, the simplified model includes the substrate and the transmission line trace arranged on the substrate; setting the dielectric constant of the substrate as a variable, performing parameter scanning simulation, and obtaining the curve of the corresponding phase shift value of the set frequency as a function of the dielectric constant; and according to the relationship curve of frequency with phase shift value of the real model and the curve of the corresponding phase shift value of the set frequency as a function of the dielectric constant obtained by the simplified model, determining the dielectric constant value when the phase shift value of the simplified model equals the phase shift value of the real model, and using this dielectric constant value as the effective dielectric constant of the substrate including glass fibers.

In some implementations, the step of constructing the real model includes: setting parameters of the glass fiber-woven layer; constructing a stacked model of the substrate and the transmission line trace; and performing circuit design to form the required traces or patterns.

In some implementations, the glass fiber-woven layer includes interwoven longitudinal glass fibers and transverse glass fibers, and the parameters of the glass fiber-woven layer include widths, spacings, heights, thicknesses, dielectric constants, and dielectric loss factors of the longitudinal glass fibers and the transverse glass fibers.

In some implementations, a dielectric layer is arranged on the top surface of the substrate and covers the transmission line trace, and the dielectric layer and the substrate jointly cover the transmission line trace, and the bottom surface of the substrate is provided with a lower ground plane layer, and the step of constructing a stacked model of the substrate and the transmission line trace includes: setting the height, dielectric constant, and dielectric loss factor of the dielectric layer and the substrate, respectively; and setting the height of the transmission line trace and the height of the lower ground plane layer.

In some implementations, the transmission line trace is a microstrip line or a stripline; a) when the transmission line trace is the microstrip line, the dielectric layer is a solder mask layer; and b) when the transmission line trace is the stripline, the dielectric layer is a resin material layer, and the step of setting the height of the transmission line trace and the height of the lower ground plane layer further includes: setting the height of the upper ground plane layer.

In some implementations, the step of performing phase shift value simulation on the real model includes: simulating the circuit to obtain the frequency and the corresponding phase shift value of the frequency; and obtaining the relationship curve of the frequency with the phase shift value through frequency parameter scanning.

In some implementations, the step of constructing a simplified model includes: constructing a simplified model, the simplified model maintains the same stacked structure and wiring as the real model, and the simplified model does not include the glass fiber-woven layer.

The present disclosure further provides an apparatus, including a memory, a processor, and a computer program stored on the memory, and the processor executes the computer program to achieve the steps of the aforementioned method.

The present disclosure further provides a computer-readable storage medium storing a computer program thereon, and when executed by a processor, the computer program performs the steps of the aforementioned method.

### BRIEF DESCRIPTION OF THE DRAWINGS

FIG. 1 is a schematic diagram of the steps of a method for characterizing the effective dielectric constant of a substrate including glass fibers provided by a specific implementation of the present disclosure;

FIG. 2A is a top view schematic diagram of a glass woven fiber layer provided by a specific implementation of the present disclosure;

FIG. 2B is a cross-sectional view schematic diagram along line A-A1 in FIG. 2A;

FIG. 3A is a schematic diagram of a stacked model provided by a specific implementation of the present disclosure;

FIG. 3B is a schematic diagram of another stacked model provided by another specific implementation of the present disclosure;

FIG. 4 is a relationship curve of frequency with phase shift value; and

FIG. 5 is a relationship curve of phase shift value with dielectric constant corresponding to a frequency of 40 GHz.

### DETAILED DESCRIPTION

The specific implementation of the method for characterizing the effective dielectric constant of a substrate including glass fibers, the apparatus, and the computer-readable storage medium, provided by the present disclosure are described in detail below in conjunction with the accompanying drawings.

The dielectric properties of materials directly affect the propagation speed, signal integrity, and transmission loss of signals. Consequently, a measurement of the dielectric properties is crucial for package design, integrated circuit (IC) design, and printed circuit board (PCB) design. Wherein the dielectric constant (Dk) is one of the primary parameters of the dielectric properties of materials. The dielectric constant, on the one hand, affects the impedance matching of signal lines, and on the other hand, determines propagation speed and phase delay of signals.

For substrates including glass fibers, the glass fiber-woven structures determine that their simulation requires finer mesh division, particularly during the process of parameter optimization, which can result in a significant computational burden. Moreover, a simulation in complex circuit models including glass fiber-woven structures will face issues of increased simulation time and low computational efficiency. In order to address these issues, the equivalent dielectric constant (ε_eff) is commonly used to characterize the characteristics of substrates including glass fibers. Accurate dielectric constant data is crucial for achieving consistency of the model with measurement results. Without an accurate dielectric material model, a closed loop of consistency of simulation with actual performance cannot be realized.

The equivalent dielectric constant of substrate materials will vary with the resin thickness, glass fiber characteristics, and fiber weaving mode. Taking the typical substrate material GHPL-970LF as an example, its glass fiber types include # 1027, # 1037, # 1067, #1078, # 1280, and # 2116, etc., and some glass fiber types also have different thicknesses. Different material combinations and geometric structures will generate unique dielectric constant values, bringing additional challenges for accurate modeling and prediction. In testing and characterization, there are also challenges such as diversified usage scenarios, measurement errors, and differences in specific application circuits. Diversified scenarios: the effective dielectric constant of substrate materials is significantly affected by changes in the weaving ratio and pattern of glass fibers. Physical testing characterization for every possible combination requires a significant amount of time, cost, and effort. Measurement error: processing tolerances and surface roughness of conductors during sample manufacturing may introduce errors, thereby affecting the measurement accuracy of dielectric properties. Differences in specific application circuits: dielectric constants obtained from specific test structures (e.g., microstrip line characterization is more suitable for microstrip lines, which are field structure-related) typically only exhibit high accuracy in configurations and stacks similar to the test design. This specificity limits the universality and applicability of test results in different designs or material settings.

Therefore, in methods for characterizing the effective dielectric constant of a substrate including glass fibers, how to solve the limitations of traditional testing methods, while ensuring high accuracy for specific applications has become a current research focus.

The method for characterizing the effective dielectric constant of a substrate including glass fibers provided by the specific implementations of the present disclosure bridges the gap between simulation accuracy and practical feasibility, and provides a robust solution available for characterizing the dielectric properties of package materials including glass fibers. In the method for characterizing the effective dielectric constant of a substrate including glass fibers provided by the specific implementations of the present disclosure, by comparing the actual truncated trace model with the simplified model, an effective dielectric material model is obtained, and this method is simple and efficient; it ensures accurate extraction of effective dielectric properties and is consistent with actual application scenarios; it can adapt to different glass fiber patterns and resin compositions, and has universality.

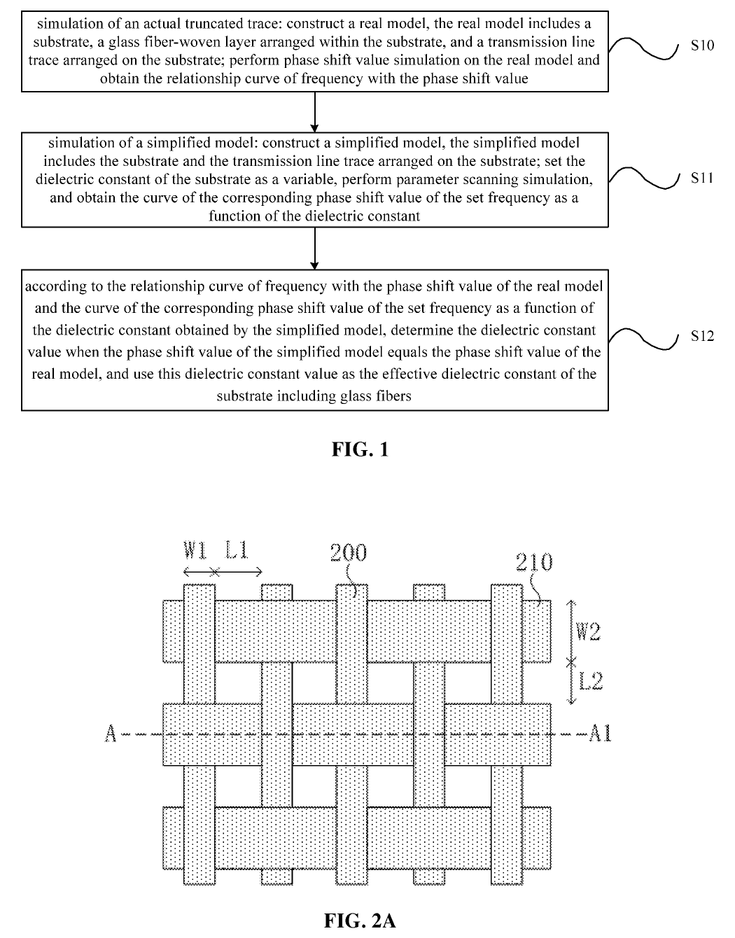

FIG. 1 is a schematic diagram of the steps of the method for characterizing the effective dielectric constant of a substrate including glass fibers provided by a specific implementation of the present disclosure, referring to FIG. 1, the method for characterizing the effective dielectric constant of a substrate including glass fibers includes:

Step S10: simulation of an actual truncated trace: constructing a real model, the real model includes a substrate, a glass fiber-woven layer arranged within the substrate, and a transmission line trace arranged on the substrate; performing phase shift value simulation on the real model and obtaining the relationship curve of frequency with the phase shift value.

In some embodiments, in this step, a real model including the glass fiber-woven layer is constructed, and parameters of the real model can be set according to design requirements. The material of the substrate may be a resin material.

In the present specific implementation, the step of constructing the real model includes:

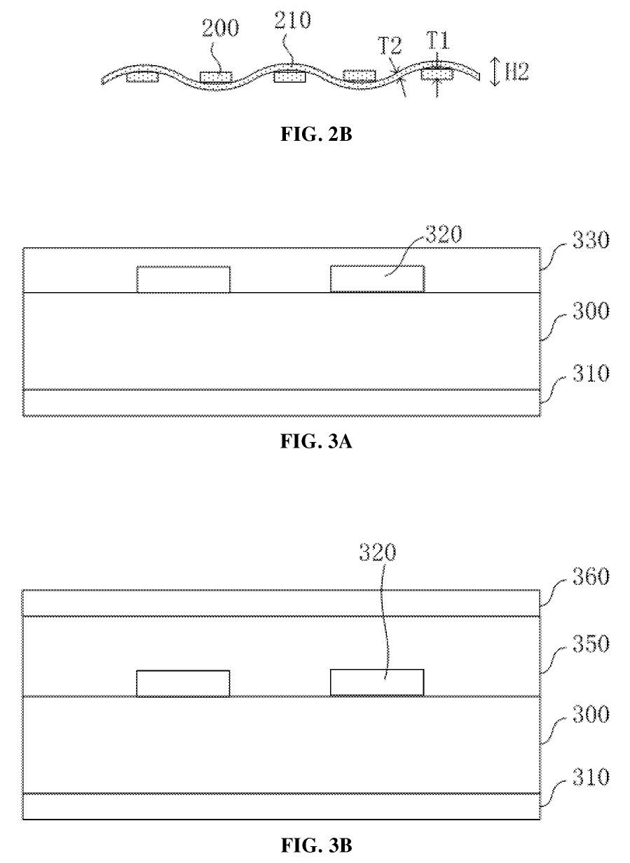

Setting the parameters of the glass fiber-woven layer, i.e., defining the geometric characteristics of the glass fiber-woven layer. FIG. 2A is a top view schematic diagram of a glass woven fiber layer provided by a specific implementation of the present disclosure, FIG. 2B is a cross-sectional view schematic diagram along line A-A1 in FIG. 2A. As shown in FIGS. 2A and 2B, the glass fiber-woven layer includes interwoven longitudinal glass fiber 200 and transverse glass fiber 210, and the parameters of the glass fiber-woven layer include widths, spacings, heights, thicknesses, dielectric constants (DKs), and dielectric loss factors (DFs) of the longitudinal glass fiber 200 and the transverse glass fiber 210. In some embodiments, the parameters of the glass fiber-woven layer include the width W1, spacing L1, height (not shown in the figure), thickness T1, dielectric constant, and dielectric loss factor of the longitudinal glass fiber 200. Wherein the width of longitudinal glass fiber 200 refers to the dimension of a longitudinal glass fiber 200 in a vertical direction of the extension direction of the longitudinal glass fiber 200, and the spacing of longitudinal glass fiber 200 refers to the distance between two adjacent longitudinal glass fibers 200, and the height of longitudinal glass fiber 200 refers to the distance between the wave crest and wave trough of a longitudinal glass fiber 200 in the extension direction of the longitudinal glass fiber 200, and the thickness of longitudinal glass fiber 200 refers to the width of a cross-section of a longitudinal glass fiber 200. The parameters of the glass fiber-woven layer include the width W2, spacing L2, height H2, thickness T2, dielectric constant, and dielectric loss factor of the transverse glass fiber 210. The width of transverse glass fiber 210 refers to the dimension of a transverse glass fiber 210 in a vertical direction of the extension direction of the transverse glass fiber 210, and the spacing of transverse glass fiber 210 refers to the distance between two adjacent transverse glass fiber 210, and the height of transverse glass fiber 210 refers to the distance between the wave crest and wave trough of a transverse glass fiber 210 in the extension direction of the transverse glass fiber 210, and the thickness of transverse glass fiber 210 refers to the width of a cross-section of a transverse glass fiber 210.

Constructing the stacked model of the substrate and the transmission line trace, i.e., defining the parameters of the substrate and transmission line trace in the real model. FIG. 3A is a schematic diagram of a stacked model provided by a specific implementation of the present disclosure, FIG. 3B is a schematic diagram of another stacked model provided by another specific implementation of the present disclosure. The bottom surface of the substrate 300 is provided with a lower ground plane layer 310 for grounding, and the transmission line trace 320 is arranged on the top surface of the substrate 300. A dielectric layer is arranged on the top surface of the substrate 300 and covers the transmission line trace 320, and the dielectric layer and the substrate 300 jointly cover the transmission line trace 320.

In some implementations, the transmission line trace 320 is a microstrip line or a stripline. When the transmission line trace 320 is the microstrip line, the dielectric layer is a solder mask layer 330, as shown in FIG. 3A, the transmission line trace 320 is arranged on the top surface of the substrate 300, and the solder mask layer 330 covers the transmission line trace 320. In FIG. 3A, the microstrip line is a symmetrically coupled microstrip line, and the microstrip line is a strip-shaped trace attached to the substrate surface. The main component portion of the microstrip line is a single-layer and double-layer metal track, which is narrow in width and relatively thin in thickness. When the transmission line trace 320 is the stripline, the top surface of the dielectric layer is also provided with an upper ground plane layer 360, and the dielectric layer is a resin material layer 350, as shown in FIG. 3B, the transmission line trace 320 is arranged on the top surface of the substrate 300, and the resin material layer 350 covers the transmission line trace 320. In FIG. 3B, the stripline is a symmetrically coupled stripline, and the stripline is a strip-shaped trace embedded within the dielectric layer, which is embedded between the lower ground plane layer 310 on the bottom surface of the substrate 300 and the upper ground plane layer 360 on the top surface of the resin material layer 350.

The step of constructing the stacked model of the substrate and the transmission line trace includes: setting the height, dielectric constant, and dielectric loss factor of the dielectric layer and the substrate, respectively; setting the height of the transmission line trace and the height of the lower ground plane layer; and when the transmission line trace is a stripline, it also includes setting the height of the upper ground plane layer. In some specific implementations, the dielectric constants and the dielectric loss factors of the substrate and the dielectric layer are the same. It can be understood that other parameters related to the transmission line trace may also be defined in this step to provide a stacked model that can be used for simulation.

After the step of constructing the stacked model of the substrate and transmission line trace, circuit design is carried out to form the required traces or patterns. In this step, circuit design is carried out according to the transmission line trace in the stacked model.

The step of constructing the real model ends with the completion of circuit design.

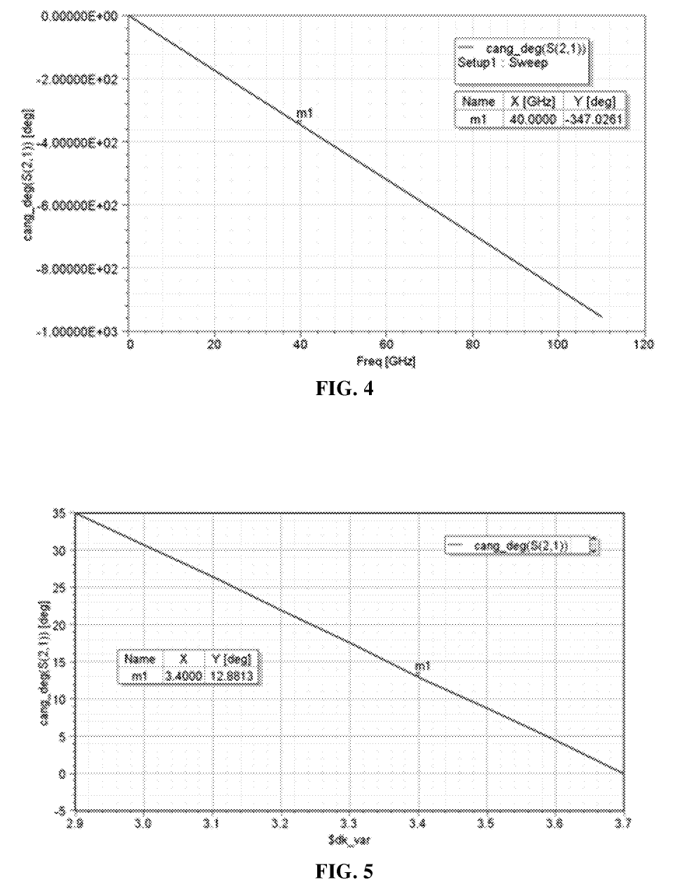

After the step of constructing the real model, a phase shift value simulation is performed on the real model to obtain the relationship curve of frequency with phase shift value. In this step, simulation software may be used to perform the phase shift value simulation on the real model to calculate the phase shift values at specific frequencies, so that the relationship curve of frequency with phase shift value is obtained. In some implementations, the step of performing phase shift value simulation on the real model includes: simulating the circuit to obtain the frequency and the corresponding phase shift value of this frequency, and obtaining the relationship curve of frequency with phase shift value through frequency parameter scanning, and referring to FIG. 4, it is the relationship curve of frequency with phase shift value.

Referring again to FIG. 1, after performing the simulation of the actual truncated trace (step S10), the method includes: step S11, simulation of a simplified model: constructing a simplified model, and the simplified model includes the substrate and the transmission line trace arranged on the substrate; setting the dielectric constant of the substrate as a variable, performing parameter scanning simulation, and obtaining the curve of the corresponding phase shift value of the set frequency as a function of the dielectric constant.

In this step, the difference between the simplified model and the real model lies in that the glass fiber-woven layer is not provided in the simplified model, all parameters of the substrate in the simplified model and of the substrate in the real model are the same, and all parameters of the transmission line trace in the simplified model and of the transmission line trace in the real model are the same. The step of constructing a simplified model includes constructing a simplified model, the simplified model maintains the same stacked structure and wiring as the real model, and the simplified model does not include the glass fiber-woven layer. In some implementations, the method for constructing the simplified model is the same as the method for constructing the stacked model; in some embodiments, the simplified model is obtained by deleting the glass fiber-woven layer on the basis of the real model.

After constructing the simplified model, the dielectric constant of the substrate in the simplified model is set as a variable, and parameter scanning simulation is performed on the simplified model to obtain the curve of the corresponding phase shift value of the set frequency as a function of the dielectric constant, and referring to FIG. 5, it is a relationship curve of phase shift value with dielectric constant corresponding to a frequency of 40 GHz. Wherein simulation software can be used to perform a parameter scanning simulation on the simplified model. A frequency corresponds to a relationship curve of phase shift value with dielectric constant, in this step, a plurality of frequencies and a relationship curve of corresponding phase shift value and dielectric constant of each frequency can be obtained.

In some embodiments, the simulation software used in steps S10 and S11 is the transmission line port (i.e., High Frequency Structure Simulator (HFSS)) simulation software.

Referring again to FIG. 1, after performing the simulation of the simplified model (step S11), the method includes: step S12, according to the relationship curve of frequency with the phase shift value of the real model and the curve of the corresponding phase shift value of the set frequency as a function of the dielectric constant obtained by the simplified model, determining the dielectric constant value when the phase shift value of the simplified model equals the phase shift value of the real model, and using this dielectric constant value as the effective dielectric constant of the substrate including glass fibers.

In this step, the relationship curve of frequency with phase shift value of the real model is associated with the curve of the corresponding phase shift value of the set frequency as a function of the dielectric constant obtained by the simplified model to obtain the effective dielectric constant of the substrate including glass fibers. For example, as shown in FIG. 4, at point m1, the frequency is 40 GHz, and the phase shift value is -347°. As shown in FIG. 5, in the curve corresponding to the frequency of 40 GHz, when the dielectric constant (Dk) is 3.4, the phase shift value of the simplified model is 12.88°. Since phase shifts are typically expressed between -180° and 180°. By subtracting 360° from 12.88°, it yields a phase shift value of -347.02°, and this phase shift value matches the phase shift value at point m1. Therefore, the actual effective dielectric constant (ε_eff) of the substrate including glass fibers is 3.4.

The method for characterizing the effective dielectric constant of a substrate including glass fibers provided by the specific implementations of the present disclosure bridges the gap between simulation accuracy and practical feasibility, and provides a robust solution available for characterizing the dielectric properties of package materials including glass fibers. In the method for characterizing the effective dielectric constant of a substrate including glass fibers provided by the specific implementations of the present disclosure, by comparing the actual truncated trace model with the simplified model, an effective dielectric material model is obtained, and this method is simple and efficient; it ensures accurate extraction of effective dielectric properties and is consistent with actual application scenarios; it can adapt to different glass fiber patterns and resin compositions, and has universality.

For example, for full-scale simulation of complex high-density traces on models including glass fibers, the existing method is to use 3D electromagnetic simulation tools (e.g., HFSS, CST) to model and simulate large-scale designs including glass fiber-woven structures. Its limitation lies in the high demand for computing resources, due to the complex geometric shape of glass fiber, fine mesh division is required, which increases simulation time and memory usage, resulting in excessively long running times and even inability to complete simulations; the scalability is poor, and it is not suitable for iterative design processes or real-time optimization. By utilizing a simplified equivalent model, the method for characterizing the effective dielectric constant of a substrate including glass fibers provided by the specific implementations of the present disclosure avoids the need for detailed mesh division and maintains accuracy, while reducing the demand for computational resources.

For another example, some simulation methods are used to design and manufacture test vehicle (TV) structures (e.g., microstrip lines or resonators), and measure dielectric properties using devices such as vector network analyzers (VNAs), etc. Its limitations lie in the high cost and time cycle, making it very expensive and time-consuming to manufacture test TVs for different scenarios (such as different resin to fiber ratios or fabric patterns); measurement errors, due to reasons such as processing tolerances and surface roughness, etc., may lead to accuracy errors; and the universality is limited, test results typically are circuit structures configured with specific field distributions, which may not be directly applicable to other stacks or circuit designs. By using a simulation-based extraction method, the method for characterizing the effective dielectric constant of a substrate including glass fibers provided by the specific implementations of the present disclosure avoids the need for a large number of TV productions; by integrating this method into design-specific simulations, the universality of the results is ensured.

For another example, in some simulation methods, design of experiments (DOE) is applied, and the design of experiments is a statistical method used to optimize a plurality of parameters. By systematically changing parameters such as resin thickness, glass fabric pattern, and trace dimensions, etc., it identifies combinations that can provide optimal performance metrics. Its limitation lies in that each parameter combination typically requires physical prototyping or extensive simulation iterations, which leads to increased time and cost; root cause is that insights are limited, and although DOE can identify trends and correlations, it rarely clearly identifies specific factors that lead to performance deviations; the trial-and-error process is complex, and the lack of deterministic analysis means that new product designs often need to start from scratch, which makes that the research and development of each new product is a difficult attempt and requires substantial effort to find suitable solutions. The method for characterizing the effective dielectric constant of a substrate including glass fibers provided by the specific implementations of the present disclosure is different from DOE, the present disclosure is based on a simulation method to directly associate the effective dielectric constant with physical parameters, thereby enabling identification of the root cause of performance deviations; reliance on prototyping is reduced, by utilizing precise simulation tools, the need for iterative prototyping is minimized and time and resources are saved; this method has good predictive ability and can extract the dielectric constant of specific applications to achieve predictive modeling, thereby accelerating design iterations and improving accuracy.

The present disclosure further provides an apparatus including a memory, a processor, and a computer program stored on the memory, and the processor executes the computer program to achieve the steps of the aforementioned method.

The present disclosure further provides a computer-readable storage medium storing thereon a computer program, and when executed by a processor, the computer program performs the steps of the aforementioned method.

Those ordinary skilled in the art will appreciate that implementing all or part of the processes in the aforementioned embodiments may be accomplished by instructing relevant hardware through a computer program, and the computer program may be stored on a non-volatile computer-readable storage medium, and when executed, the computer program may include processes of the embodiments of the aforementioned methods. Wherein any reference to memory, storage, database or other media used in the embodiments provided by the present disclosure may include non-volatile and/or volatile memory. Non-volatile storage may include read-only memory (ROM), programmable ROM (PROM), electrically programmable ROM (EPROM), electrically erasable programmable ROM (EEPROM), or flash memory. Volatile memory may include random access memory (RAM) or external cache memory. As an illustration and not a limitation, RAM is available in various forms, such as static RAM (SRAM), dynamic RAM (DRAM), synchronous DRAM (SDRAM), double data rate SDRAM (DDRSDRAM), enhanced SDRAM (ESDRAM), synchronous link DRAM (SLDRAM), Rambus direct RAM (RDRAM), direct Rambus dynamic RAM (DRDRAM), and Rambus dynamic RAM (RDRAM), etc.

It should be noted that the terms "include" and "have" and their variations referred to in the document of the present disclosure are intended to cover non-exclusive inclusions. The terms such as "first," "second," etc., are used to distinguish similar objects and are not necessarily used to describe a specific order or sequence, and it is appreciated that unless otherwise indicated in the context clearly, the data used in this way can be interchanged in appropriate circumstances. The term "one or more" is at least partially dependent on the context and may be used to describe features, structures, or characteristics in a singular sense or in a plural sense. The term "according to" may be understood as not necessarily intended to express a set of exclusive factors, but may alternatively, also be at least partially dependent on the context, permit the existence of other factors that may not be explicitly described. In addition, the embodiments of the present disclosure and features in the embodiments may be combined with each other without conflict. Additionally, in the above explanation, descriptions of well-known components and technologies have been omitted to avoid unnecessary confusion of the concepts of the present disclosure. In the above embodiments, each embodiment focuses on illustrating differences from other embodiments, and for the same/similar parts between the embodiments, a mutual reference can be made.

The above is only the embodiments of the present disclosure, it should be noted that those ordinary skilled in the art may also make several improvements and refinements without departing from the principles of the present disclosure, and these improvements and refinements should also be considered as the protection scope of the present disclosure.

What is claimed is:

1. A method for characterizing an effective dielectric constant of a substrate comprising glass fibers, comprising:
performing a simulation on a model of an actual truncated trace comprising:
constructing a real model, wherein the real model comprises the substrate, a glass fiber-woven layer arranged within the substrate, and a transmission line trace arranged on the substrate;
performing a phase shift simulation on the real model according to a phase shift value; and
obtaining a relationship curve of frequency with the phase shift value;
performing a simulation on a simplified model comprising:
constructing the simplified model, wherein the simplified model comprises the substrate, and the transmission line trace arranged on the substrate;
setting a dielectric constant of the substrate as a variable;
performing parameter scanning simulation; and
obtaining a curve of a corresponding phase shift value of a set frequency as a function of the dielectric constant; and
according to the relationship curve of frequency with the phase shift value obtained by the real model and the curve of the corresponding phase shift value of the set frequency as the function of the dielectric constant obtained by the simplified model, determining a dielectric constant value when the phase shift value of the simplified model equals the phase shift value of the real model, and using the dielectric constant value as the effective dielectric constant of the substrate comprising the glass fibers.

2. The method for characterizing the effective dielectric constant of the substrate comprising the glass fibers according to claim 1, wherein the constructing the real model comprises:
setting parameters of the glass fiber-woven layer;
constructing a stacked model of the substrate and the transmission line trace; and
performing circuit design to form required traces or patterns.

3. The method for characterizing the effective dielectric constant of the substrate comprising the glass fibers according to claim 2, wherein the glass fiber-woven layer comprises interwoven longitudinal glass fibers and transverse glass fibers, and the parameters of the glass fiber-woven layer comprise widths, spacings, heights, thicknesses, dielectric constants, and dielectric loss factors of the longitudinal glass fibers and the transverse glass fibers.

4. The method for characterizing the effective dielectric constant of the substrate comprising the glass fibers according to claim 2, wherein a dielectric layer is arranged on a top surface of the substrate and covers the transmission line trace, and wherein a bottom surface of the substrate is provided with a lower ground plane layer, and wherein the constructing the stacked model comprises:
setting a height, a dielectric constant, and a dielectric loss factor of the dielectric layer and the substrate, respectively; and
setting a height of the transmission line trace and a height of the lower ground plane layer.

5. The method for characterizing the effective dielectric constant of the substrate comprising glass fibers according to claim 4, wherein the transmission line trace is a microstrip line or a stripline, and wherein:
a) when the transmission line trace is the microstrip line, the dielectric layer is a solder mask layer; or
b) when the transmission line trace is the stripline, the dielectric layer is a resin material layer, and wherein constructing the stacked model further comprises providing an upper ground plane layer on a top surface of the dielectric layer, and wherein the setting the height of the transmission line trace and the height of the lower ground plane layer further comprises setting a height of the upper ground plane layer.

6. The method for characterizing the effective dielectric constant of the substrate comprising glass fibers according to claim 2, wherein performing phase shift value simulation on the real model comprises:
simulating a circuit corresponding to the real model to obtain a frequency and a corresponding phase shift value; and
obtaining the relationship curve of frequency with the phase shift value through frequency parameter scanning.

7. The method for characterizing the effective dielectric constant of the substrate comprising glass fibers according to claim 1, wherein constructing the simplified model comprises: constructing a model that maintains a same stacked structure and wiring as the real model but excludes the glass fiber-woven layer.

8. An apparatus, comprising:
a memory storing computer-readable instructions; and
a processor coupled to the memory and configured to execute the computer-readable instructions, wherein the computer-readable instructions, when executed by the processor, cause the processor to perform a method for characterizing an effective dielectric constant of a substrate comprising glass fibers, wherein the method comprises:
performing a simulation on a model of an actual truncated trace comprising:
constructing a real model, wherein the real model comprises: the substrate, a glass fiber-woven layer arranged within the substrate, and a transmission line trace arranged on the substrate;
performing a phase shift simulation on the real model according to a phase shift value; and
obtaining a relationship curve of frequency with the phase shift value;
performing a simulation on a simplified model comprising:
constructing the simplified model, wherein the simplified model comprises: the substrate, and the transmission line trace arranged on the substrate;
setting a dielectric constant of the substrate as a variable;
performing parameter scanning simulation; and
obtaining a curve of a corresponding phase shift value of a set frequency as a function of the dielectric constant; and
according to the relationship curve of frequency with the phase shift value obtained by the real model and the curve of the corresponding phase shift value of the set frequency as the function of the dielectric constant obtained by the simplified model, determining a dielectric constant value when the phase shift value of the simplified model equals the phase shift value of the real model, and using the dielectric constant value as the effective dielectric constant of the substrate comprising the glass fibers.

9. The apparatus according to claim 8, wherein the constructing the real model comprises:
setting parameters of the glass fiber-woven layer;
constructing a stacked model of the substrate and the transmission line trace; and
performing circuit design to form required traces or patterns.

10. The apparatus according to claim 9, wherein the glass fiber-woven layer comprises interwoven longitudinal glass fibers and transverse glass fibers, and the parameters of the glass fiber-woven layer comprise widths, spacings, heights, thicknesses, dielectric constants, and dielectric loss factors of the longitudinal glass fibers and the transverse glass fibers.

11. The apparatus according to claim 9, wherein a dielectric layer is arranged on a top surface of the substrate and covers the transmission line trace, and wherein a bottom surface of the substrate is provided with a lower ground plane layer, and wherein the constructing the stacked model comprises:
setting a height, a dielectric constant, and a dielectric loss factor of the dielectric layer and the substrate, respectively; and
setting a height of the transmission line trace and a height of the lower ground plane layer.

12. The apparatus according to claim 11, wherein the transmission line trace is a microstrip line or a stripline, and wherein:
a) when the transmission line trace is the microstrip line, the dielectric layer is a solder mask layer; or
b) when the transmission line trace is the stripline, the dielectric layer is a resin material layer, and wherein constructing the stacked model further comprises providing an upper ground plane layer on a top surface of the dielectric layer, and wherein the setting the height of the transmission line trace and the height of the lower ground plane layer further comprises setting a height of the upper ground plane layer.

13. The apparatus according to claim 9, wherein performing phase shift value simulation on the real model comprises:
simulating a circuit corresponding to the real model to obtain a frequency and a corresponding phase shift value; and
obtaining the relationship curve of frequency with the phase shift value through frequency parameter scanning.

14. The apparatus according to claim 8, wherein constructing the simplified model comprises: constructing a model that maintains a same stacked structure and wiring as the real model but excludes the glass fiber-woven layer.

15. A non-transitory computer-readable storage medium storing instructions that, when executed by a processor, cause the processor to perform a method for characterizing an effective dielectric constant of a substrate comprising glass fibers, wherein the method comprises:
performing a simulation on a model of an actual truncated trace comprising:
constructing a real model, wherein the real model comprises: the substrate, a glass fiber-woven layer arranged within the substrate, and a transmission line trace arranged on the substrate;
performing a phase shift simulation on the real model according to a phase shift value; and
obtaining a relationship curve of frequency with the phase shift value;
performing a simulation on a simplified model comprising:
constructing the simplified model, wherein the simplified model comprises: the substrate, and the transmission line trace arranged on the substrate;
setting a dielectric constant of the substrate as a variable;
performing parameter scanning simulation; and
obtaining a curve of a corresponding phase shift value of a set frequency as a function of the dielectric constant; and
according to the relationship curve of frequency with the phase shift value obtained by the real model and the curve of the corresponding phase shift value of the set frequency as the function of the dielectric constant obtained by the simplified model, determining a dielectric constant value when the phase shift value of the simplified model equals the phase shift value of the real model, and using the dielectric constant value as the effective dielectric constant of the substrate comprising the glass fibers.

16. The non-transitory computer-readable storage medium according to claim 15, wherein the constructing the real model comprises:
setting parameters of the glass fiber-woven layer;
constructing a stacked model of the substrate and the transmission line trace; and
performing circuit design to form required traces or patterns.

17. The non-transitory computer-readable storage medium according to claim 16, wherein the glass fiber-woven layer comprises interwoven longitudinal glass fibers and transverse glass fibers, and the parameters of the glass fiber-woven layer comprise widths, spacings, heights, thicknesses, dielectric constants, and dielectric loss factors of the longitudinal glass fibers and the transverse glass fibers.

18. The non-transitory computer-readable storage medium according to claim 16, wherein a dielectric layer is arranged on a top surface of the substrate and covers the transmission line trace, and wherein a bottom surface of the substrate is provided with a lower ground plane layer, and wherein the constructing the stacked model comprises:
setting a height, a dielectric constant, and a dielectric loss factor of the dielectric layer and the substrate, respectively; and
setting a height of the transmission line trace and a height of the lower ground plane layer.

19. The non-transitory computer-readable storage medium according to claim 18, wherein the transmission line trace is a microstrip line or a stripline, and wherein:
a) when the transmission line trace is the microstrip line, the dielectric layer is a solder mask layer; or
b) when the transmission line trace is the stripline, the dielectric layer is a resin material layer, and wherein constructing the stacked model further comprises providing an upper ground plane layer on a top surface of the dielectric layer, and wherein the setting the height of the transmission line trace and the height of the lower ground plane layer further comprises setting a height of the upper ground plane layer.

20. The non-transitory computer-readable storage medium according to claim 16, wherein performing phase shift value simulation on the real model comprises:
simulating a circuit corresponding to the real model to obtain a frequency and a corresponding phase shift value; and
obtaining the relationship curve of frequency with the phase shift value through frequency parameter scanning.
